(19) United States
(12) Patent Application Publication
(10) Pub. No.: US 2015/0099921 A1
(43) Pub. Date: Apr. 9, 2015

(54) TREATMENT OF DEGENERATIVE BRAIN DISORDERS USING TRANSCRANIAL MAGNETIC STIMULATION

(71) Applicant: M. Bret SCHNEIDER, Portola Valley, CA (US)

(72) Inventor: M. Bret SCHNEIDER, Portola Valley, CA (US)

(21) Appl. No.: 14/510,858

(22) Filed: Oct. 9, 2014

Related U.S. Application Data
(60) Provisional application No. 61/888,943, filed on Oct. 9, 2013.

(51) Int. Cl. A61N 2/02; A61N 2/00

(52) U.S. Cl. CPC A61N 2/02; A61N 2/006
USPC 600/14

(57) ABSTRACT

Methods and systems for transcranial magnetic stimulation applied to the posterior cingulate including bundle fibers to treat Alzheimer's. Excitatory impulses from (or triggered by) the applied TMS may be transmitted through to the entorhinal cortex and hippocampus, which are early sites in which Alzheimer's disease begins to develop. Excitatory signals in the hippocampus and entorhinal cortex strengthen connectivity between cells, and increase. Overall metabolic activity may be increased, serving to help promote resistance to the Alzheimer's disease process and encouraging maturation and integration of neural stem cells native to this region of the brain.

### CROSS REFERENCES TO RELATED APPLICATIONS

This patent application claims priority to U.S. Provisional Patent Application No. 61/888,943, titled “TREATMENT OF DEGENERATIVE BRAIN DISORDERS USING TRANSCRANIAL MAGNETIC STIMULATION” and filed on Oct. 9, 2013, the entirety of which is herein incorporated by reference in its entirety.

This application may be related to one or more of the following U.S. patent applications. U.S. patent application Ser. No. 12/679,960, filed on Sep. 15, 2010 (now U.S. Pat. No. 8,265,910); U.S. patent application Ser. No. 12/324,227, filed on Nov. 26, 2008 (now U.S. Pat. No. 8,267,850); U.S. patent application Ser. No. 13/586,640, filed on Aug. 15, 2012 (now U.S. Pat. No. 8,523,753); U.S. patent application Ser. No. 13/141,100, filed on Aug. 1, 2011 (now U.S. Pat. No. 8,723,628); U.S. patent application Ser. No. 12/912,650, filed on Oct. 26, 2010 (now U.S. Pat. No. 8,795,148); U.S. patent application Ser. No. 12/670,938, filed on Jun. 17, 2010 (published as US-2010-0256438); U.S. patent application Ser. No. 14/247,087, filed on Apr. 7, 2014; U.S. patent application Ser. No. 12/838,299, filed on Jul. 16, 2010 (published as US-2010-0286470-A1); U.S. patent application Ser. No. 13/512,496, filed on Sep. 17, 2012 (published as US-2013-0006039-A1); U.S. patent application Ser. No. 13/169,967, filed on Jun. 27, 2011 (published as US-2014-0135565-A9); U.S. patent application Ser. No. 13/808,806, filed on Apr. 23, 2013 (published as US-2013-0204330); U.S. patent application Ser. No. 13/888,263, filed on May 6, 2013 (published as US-2013-0317281-A1); U.S. patent application Ser. No. 13/877,428, filed on Jun. 27, 2013 (published as US-2013-0267763-A1); and U.S. patent application Ser. No. 14/131,223, filed on Mar. 25, 2014 (published as US-2014-0200388-A1). Each of these patents and patent applications is herein incorporated by reference in its entirety.

### BACKGROUND

Alzheimer's disease, in its fully evident form, affects some 5.4 million persons in the United States alone—about 1.8 percent of the population. It is responsible for direct medical costs of $172 billion per year. The total annual disease burden in the United States alone exceeds $200 billion. The worldwide prevalence is several-fold higher, and disease burden is at least twice that cited for the United States. As the demographics of the US, Japan and many western European nations shift toward greater age, the toll of Alzheimer's disease is projected to be immense. There is therefore an urgent need for novel therapeutic approaches for Alzheimer's disease.

The few medications that are FDA approved for the treatment of Alzheimer's disease, for example acetylcholinesterase inhibitors (e.g. tacrine) or NMDA receptor antagonists (e.g. memantine) have modest effects at best, typically delaying the course of cognitive decline by a few months. New, more aggressive methods of fighting Alzheimer's disease include approaches taken by Andreas Lozano and colleagues and the company Functional Neuromodulation Inc. In this model, electrical stimulators are neurosurgically implanted in the fornix, a major fiber bundle that transmits signal to the entorhinal cortex and hippocampus. Early results appear to show increased cellular metabolism and connectivity between brain regions, as well as suggestions of cognitive preservation or improvement over a one-year time span.

It would be desirable to have safe, non-invasive means by which one could stimulate areas of the brain affected by illnesses such as Alzheimer's disease and slow the course of the disease or possibly reverse it. However, brain regions that have direct projections to the hippocampus are not readily accessible to standard non-invasive brain stimulation devices such as the NeuroStar™ system by Neuronetics Inc. (Malvern, Pa.), as the major projects to this area lie beneath the cortical surface, too deep to directly reach with single-coil rTMS.

By contrast, the we herein propose a system to non-invasively, directly and selectively stimulate deep brain regions such as the anterior cingulate bundle, and to steer induced pulsed electrical currents at depth within the brain. Because the posterior cingulate is of similar physical nature to the anterior cingulate and is surrounded by analogous cortical matter, it can also be used to selectively stimulate the posterior cingulate bundle. The posterior cingulate bundle has projections directly meeting hippocampus and entorhinal cortex, which the systems and device described herein can therefore stimulate. In response to such stimulation, existing mature neurons and their interconnections are strengthened and they sprout more axon and dendrites. Furthermore, neural progenitor cells (essentially neuronal stem cells) exposed to pulsed electrical currents may sprout axons and dendrites, connect to one another and eventually morph into adult neurons.

### SUMMARY OF THE DISCLOSURE

Described herein are methods, devices and systems for treating (e.g., preventing, treating or reducing the effect of) Alzheimer's by specific transcranial magnetic stimluation. For example, described herein are transcranial magnetic stimulation systems and methods of use for selective, non-invasive deep brain stimulation of the posterior cingulate bundle. Projections from this bundle relay the signal to the hippocampus and entorhinal cortex. Repeated treatments targeted upon the posterior cingulate bundle may increase brain metabolism in the temporal lobe increase neural interconnectivity, strengthen existing neuronal connections are strengthened, and the prompt the maturation and integration of neural and glial stem cells. This may be used to impede or reverse the progression of Alzheimer's disease. A similar approach may be used to address other degenerative brain conditions and brain injury conditions including traumatic brain injury and post-stroke recovery.

For example, described herein are methods of treating Alzheimer's by non-invasively stimulating brain areas involved in Alzheimer's disease pathology, and particularly the posterior cingulate nerve fiber bundle (referred to herein as the posterior cingulate). For example, the method may include: orienting and/or positioning a plurality of transcranial magnetic stimulation (TMS) coils around a back of a patient's head to direct TMS stimulation towards the patient's posterior cingulate; applying a rapid TMS pulse pattern (e.g., of greater than 10 Hz) from each of the plurality of TMS coils (either separately/independently or jointly) to increase metabolic activity (e.g., by driving stimulation, inducing action potentials, etc.) of the patient's posterior cingulate for between about thirty seconds and 3 hours; and repeating the orienting and applying steps above for a plurality of treatment sessions. Any appropriate number of sessions may be used, including, for example, 5 or more, 10 or more, 15 or more, 20 or more, 25 or more, 30 or more, 40 or more, 50 or more, 60 or more, 70 or more, 80 or more, 90 or more, or the like. The sessions may be ongoing (e.g., continued indefinitely), or they may be for a finite amount of time. Sessions may be separated by a minimum and/or maximum intersession interval, e.g. time between sessions. For example, an intersession interval may be between about 3 hours and 30 days (e.g., 1× day, 2× day, 3× day, 4× day, 1× week, 2× week, 3× week, 4× week, 5× week, etc.). For example, the step of repeating may include repeating more than 30 sessions, more than 60 sessions, more than 90 session, etc.

In general, the step of orienting the TMS coils (which may also be referred to as TMS electromagnets or TMS magnets) may include positioning between two and six TMS coils around the patient's head at different locations to apply TMS that stimulates the subject's posterior cingulate. In particular, all or a subset of the TMS coils may be positioned around a back portion of a subject's head such as, in particular, positioning a first TMS coil and a second TMS coils (or additional TMS coils) against or adjacent to a region of the patient's head including the C_{Z}, P₃, P₂, and P₄ standard EEG 10-20 system set of locations. This is illustrated below and described.

Any appropriate TMS coil may be used, but particularly the “bent coil” or “v-shaped”, “swept-back”, “I-bottomed” or otherwise curved/bent TMS coils may be used, such as those incorporated by reference above. Such coils are of particular interest because they are sufficiently large enough to provide the high-strength magnetic fields required for TMS but may have a patient contacting surface that is sufficiently small so that multiple such coils may be positioned in the relatively small region on the back of the subject's head described herein. The coils may generally be cooled (allowing them to apply the high magnetic fields while maintaining a low temperature) and may be enclosed in an outer housing as described below.

In general, Transcranial magnetic stimulation (TMS) is a noninvasive method that causes depolarization or hyperpolarization in the neurons of the brain by electromagnetic induction that induces weak electric currents using a rapidly changing magnetic field applied by the TMS coil. Thus, TMS uses a magnet instead of an electrical current to activate the brain. TMS is achieved by quickly discharging current (e.g., from a large capacitor) into a coil to produce pulsed magnetic fields of, e.g., 1-10 mT. The magnetic flux density pulse generated by the current pulse through the coil causes an electric field at the cells within the brain (e.g., the membrane of a neuron).

The step of applying a rapid TMS pulse pattern for treatment of Alzheimer's may comprise, for example, stimulating the posterior cingulate and directly or indirectly resulting in stimulation along efferents going to the patient's subiculum and dentate regions. The application of TMS may include independently applying pulsed TMS from each of two or more TMS coils oriented around a back region of the subject's head to evoke stimulation of the subject's posterior cingulate.

Applying TMS may include applying pulsed TMS at greater than 10 Hz from each of two or more (e.g., 3, 4,5, or 6) TMS coils oriented around a back region of the subject's head to neuromdulate the subject's posterior cingulate nerve fiber bundle until action potentials are propagated into the subject's entorhinal cortex and hippocampus to increase neuronal activity therein.

In general, each of the TMS coils may be independently controlled and operated. For example, each of the TMS coils (or subsets of the coils) may be triggered separately. Each of the TMS coils may be connected to a separate energy source (e.g., capacitor or bank of capacitors). A single control unit may be used to control stimulation which may include one or more pulse generators and/or oscillators to provide the rapidly-changing energy driving the magnetic field.

The plurality of TMS coils may be separately or jointly positioned. Once in position, the coils may be locked in place. For example, positioning may include locking two or more TMS coils into a position around a back of the subject's head to aim stimulation from the plurality of TMS coils to the subject's posterior cingulate nerve fiber bundle. In some variations a gantry or holder (helmet, frame, etc.) may be used to at least initially roughly position the TMS coils around the patient's head near the correct location; fine adjustment may then be made to more precisely position the TMS coils.

The step of applying the rapid TMS pulse pattern may be configured to produce a net increase in metabolic activity in the subject's posterior cingulate fiber bundle. Similarly, the step of applying the rapid TMS pulse pattern may be configured to produce an increase in metabolic activity in the temporal lobe. Thus, for example, stimulation may be controlled to produce these changes in metabolism (e.g., increasing metabolism above baseline) in the posterior cingulate and/or temporal lobe.

Applying the rapid TMS pulse pattern from each of the plurality of TMS coils may comprise applying a rapid TMS pulse pattern for any appropriate time (e.g., between 0.5 min and 3 hours, between about one minute and 1 hour, etc.). The frequency of the applied energy may be constant or varied (e.g., between about 10 Hz and 150 Hz, greater than 10 Hz, greater than 15 Hz, greater than 20 Hz, etc.). As mentioned, this rate may vary during stimulation. In some variations the rate may vary between different TMS coils. In some variations the overall rate of TMS stimulation applied is an ensemble of the TMS stimulation applied by all of the TMS coils in the plurality.

The method described herein may be used to treat any patient at risk for Alzheimer's or afflicted (e.g., positively diagnosed) with Alzheimer's. Thus, the patient may be at risk because of age, socioeconomic status, genetic background, and/or because the patient displays symptoms attributable to anything from a mild cognitive impairment to early dementia.

In some examples, a method for treating Alzheimer's by non-invasively stimulating brain areas involved in Alzheimer's disease pathology may include: positioning a first transcranial magnetic stimulation (TMS) coil and a second TMS coil against or adjacent to a back of a patient's head within a region including the C_{Z}, P₃, P₂, and P₄ standard EEG 10-20 system set of locations on the patient's head; applying a rapid TMS pulse pattern from each of the plurality of TMS coils to drive stimulation of the patient's posterior cingulate for between about thirty seconds and 3 hours; and repeating the orienting and applying steps above for a plurality of treatment sessions.

As mentioned above, positioning may comprise positioning a third TMS coil against or adjacent to the back of a patient's head (or additional TMS coils), including within the region including the C_{Z}, P₃, P₂, and P₄ standard EEG 10-20 system set of locations. Applying the rapid TMS pulse pattern may comprise applying a rapid TMS pulse pattern having a frequency of greater than 10 Hz from each of the TMS coils (or in some variations from all of the coils). For example, applying the rapid TMS pulse pattern may comprise applying pulses of TMS from each of the plurality of TMS coils.

In general, applying the rapid TMS pulse pattern comprises producing a net increase in metabolic activity in the posterior cingulate, and/or increasing metabolic activity in the patient's temporal lobe.

Another example of a method for treating Alzheimer's by non-invasively stimulating brain areas involved in Alzheimer's disease pathology includes: positioning a first transcranial magnetic stimulation (TMS) coil, a second TMS coil, and a third TMS coil against or adjacent to a back of a patient's head within a region including the C_{Z}, P₃, P₂, and P₄ standard EEG 10-20 system set of locations on the patient's head; applying a rapid TMS pulse pattern from each of the plurality of TMS coils for greater than about thirty seconds; increasing metabolic activity in the posterior cingulate; and repeating the orienting and applying steps above for a plurality of treatment sessions.

### BRIEF DESCRIPTION OF THE DRAWINGS

FIG. 1 shows a high-level schematic of key brain structures that are utilized in the methods and devices for treating Alzheimer's disease and its precursors as described herein.

FIG. 2 shows an anterior view of one embodiment of a device used within the context of the present method for treating Alzheimer's disease and its precursors described herein, having a plurality of TMS coils.

FIG. 3 is a posterior view of the embodiment shown in FIG. 2 for treating Alzheimer's disease and its precursors, including a gantry holding four TMS coils (V-shaped and/or flat-bottomed coils, each within a separate housing that is thermally regulated). Each TMS coils may be connected to a power source and controller (separately or combined), not shown.

FIG. 4 illustrates the known neuroanatomical Circuit of Papez, which provides the specific neuro-anatomical substrate upon which the present device and method may be applied.

FIG. 5 illustrates neural pathways that tie the cingulate to other brain regions, including the Circuit of Papez described in FIG. 4, plus additional connections. Stimulation of the various brain regions described herein including in particular the posterior cingulate) may be performed by stimulating one or more of the pathways connecting the posterior cingulate, so that the applied energy converges thereon; alternatively or additionally the TMS stimulation may be directly applied to the target (e.g., posterior cingulate).

FIG. 6 shows a 15-O PET image derived from use of a coil array similar to that shown in FIGS. 2 and 3, except placed over the anterior portion of the cingulate rather than the posterior portion of the cingulate. This image demonstrates significant modulation of the anterior cingulate (arrow) an relatively little cortical surface modulation. It also displays an area of modulation occurring in the posterior cingulate.

FIG. 7 is a 15-O PET image derived from use of a proprietary coil array identical to that shown in FIGS. 2 and 3, except placed over the anterior portion of the cingulate rather than the posterior portion of the cingulate. This image demonstrates significant modulation of the anterior cingulate (arrow) an relatively little cortical surface modulation. It also displays small area of modulation occurring in the posterior cingulate.

FIG. 8 is a schematic illustrating the symmetry in the anterior and posterior portions of the brain, illustrating that modulating posterior cingulate can be accomplished with only minor modification of the approach used to modulate the anterior cingulate.

FIG. 9 illustrates a standard EEG 10-20 system set of locations on the patient's head, and shows (circled) a back of a patient's head with a region including the C_{Z}, P₃, P₂, and P₄ that may be particularly useful for targeting TMS stimulation as described herein.

### DETAILED DESCRIPTION

Described herein are selective, non-invasive deep brain stimulation methods (and apparatuses for performing them) that may be used to treat, prevent, ameliorate or otherwise beneficially modify Alzheimer's disease, and particularly dementia related to Alzheimer's. Although the description included herein addresses Alzheimer's specifically, these methods and apparatuses may be use (or adapted for use) with other neurological disorders as well, including other forms of dementia and cognitive disorders. For example, Down Syndrome may share many of the same effects, including symptoms, as Alzheimer's and may also respond to the methods and apparatuses described herein.

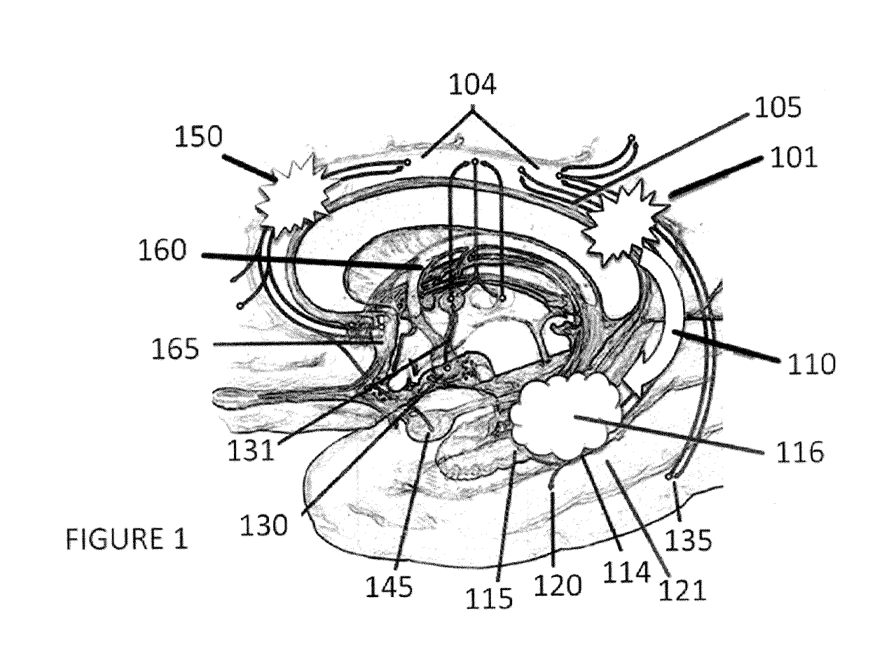

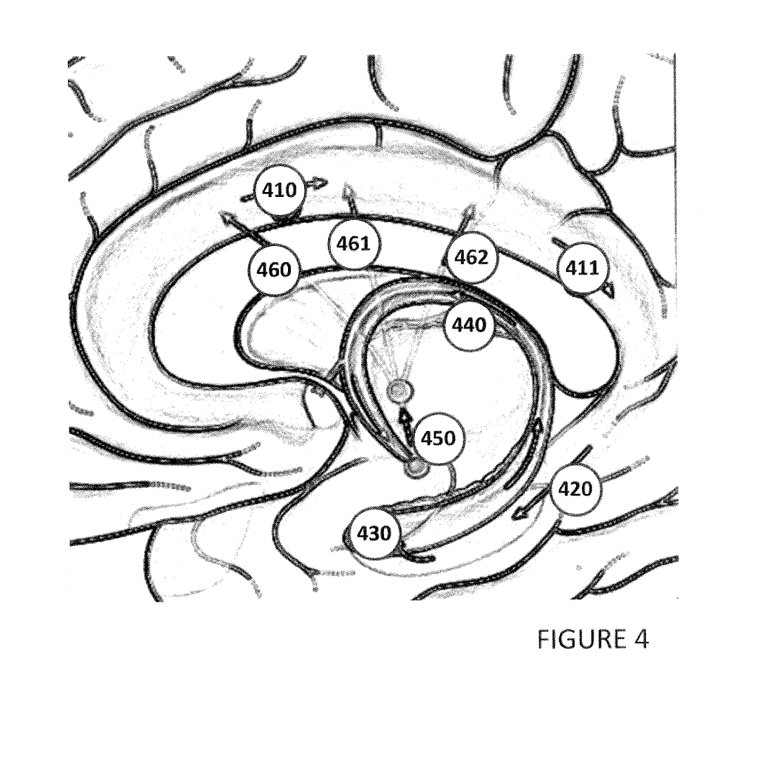

FIG. 1 illustrates a high-level schematic of key brain structures may be targeted and/or effected using the present method and devices for treating Alzheimer's disease and its precursors. These brain regions include the Circuit of Papez, which will be shown with more specificity in FIG. 4, below.

In FIG. 1, the cingulate 104 is shown. As used herein, the cingulate refers to the combination of the cingulate gyrus (gray matter) plus cingulate bundle, (AKA cingulum, a white matter tract with both anterior-extending and posterior-extending 105 portions. Posterior cingulate bundle 105 includes both afferent and efferent axons. The device and method presently described involve posterior cingulate target site 101. Posterior cingulate bundle 105 projects to cingulate-entorhinal tract 110, at which signals produced at activation site 101 are propagated into the entorhinal cortex 121 by projection 120, and into the hippocampus 115 by other projections. Other projections from posterior cingulate bundle 105 extend into subiculum 114. Pathological brain tissue 116, such as that resulting from early Alzheimer's disease processes is found in the entorhinal cortex 121 and hippocampus 115. Activation of the posterior cingulate neuronal bundle at site 101 therefore relays neuronal activation into Alzheimer's affected area 116, stimulating existing neurons and glia, and stimulating development and integration of neuronal progenitor cells in that region.

Stimulation may be further relayed up mammilo-thalamic tract 131 back to cingulate 104, thereby bolstering activity in the entire Circuit of Papez.

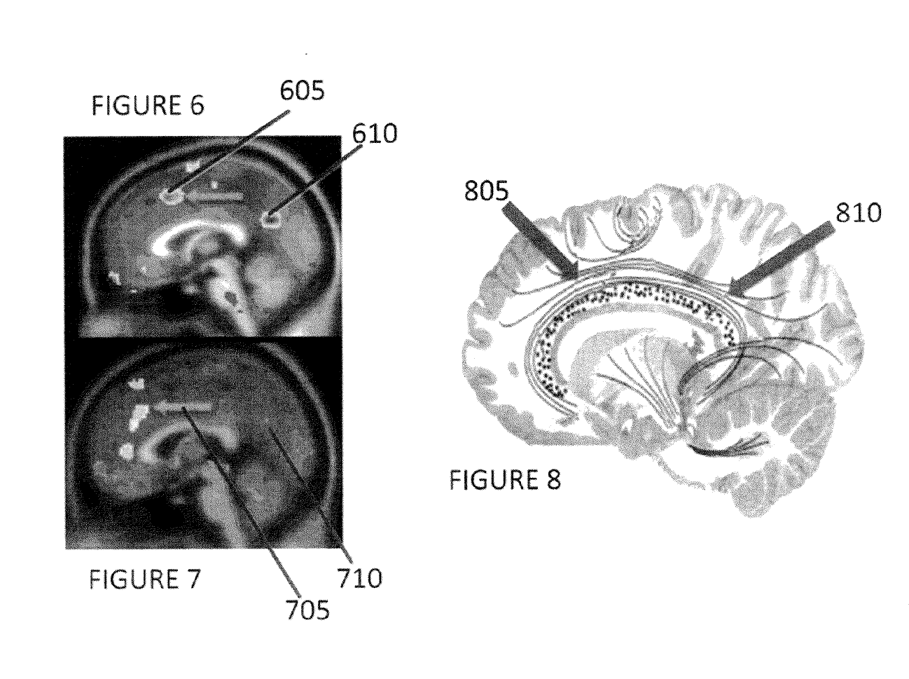

Other structures appearing in this figure, include fornix 160, which is the structure which has been shown to benefit Alzheimer's when neurosurgically (invasively) implanted with electrodes in order to relay impulses to the entorhinal cortex and hippocampus. Other structures appearing in this figure are the septal nuclei 165, Amygdala 145, and the perigenual dorsal anterior cingulate 150, a deep brain area which we have demonstrated the ability to selectively modulate, as shown below in FIGS. 6 and 7, using the multi-coil stimulation system described herein. It is anatomically quite analogous to posterior cingulate target area 101.

In general, the methods described herein modulate the posterior cingulate and therefore other regions in including the circuit of Papez, to treat Alzheimer's. As show and described herein, this is done using multiple TMS coils positioned in a target location (e.g., the back of the head, and particularly a region including the C_{Z}, P₃, P₂, and P₄ standard EEG 10-20 system set of locations on the patient's head) and applying rapid (e.g., greater than 10 Hz) TMS stimulation.

A system appropriate for this stimulation generally includes a plurality (e.g., 3, 4, 5 or 6) TMS coils that may be positioned and adjusted around the back of the patient's head to apply the appropriate TMS stimulation.

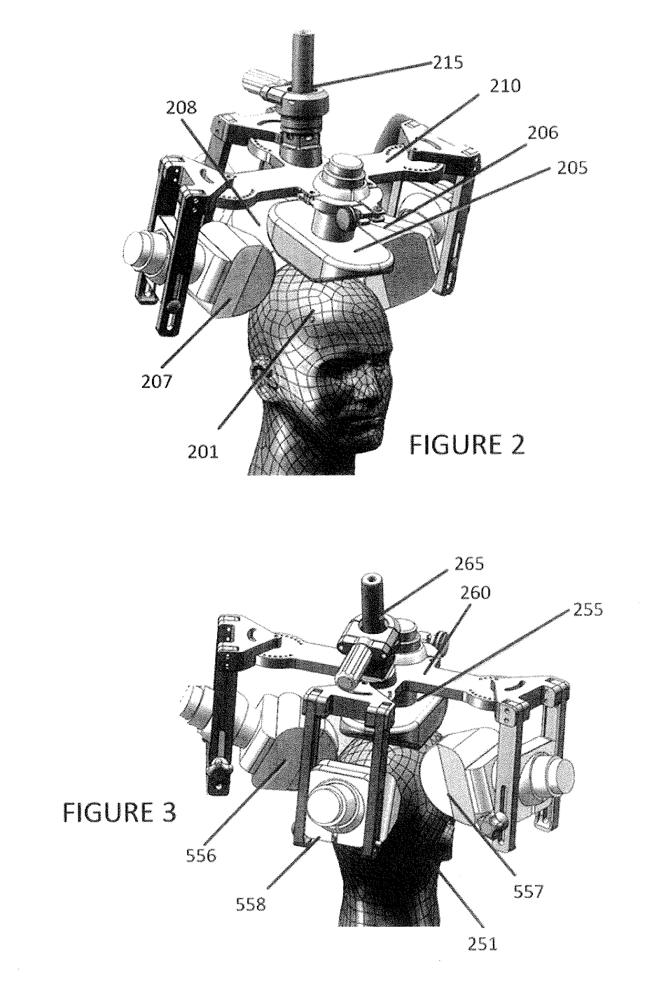

For example, FIG. 2 illustrates an anterior view of one embodiment of a device that may be used within the context of the present method for treating Alzheimer's disease and its precursor pathology. The apparatus shown in these diagrams is a 4-coil configuration (referred to herein as “Configuration B”) similar to that detailed the patent applications incorporated by reference above. In this example the system is oriented such that encompasses the posterior aspect of the head (back of the head), rather than the anterior aspect. Other coils and coil configurations including configurations of two coils and three coils produce a substantially similar effect upon the targeted brain area. In FIGS. 2 and 3, the head of patient 201 is covered by top coil 205, right side coil 207, left Side coil 206, and rear coil 208. These coils are held in the intended position by ball joints on frame 210, which is held in the proper place above the patient by frame suspension 215. The gantry shown may allow both rough and fine positioning and may lock the TMS coils in position, once they're arranged as described herein.

FIG. 3 illustrates a posterior view of the system shown in FIG. 2, which may be used within the context of the present method for treating Alzheimer's disease and its precursors. The same coil configuration as shown in FIG. 2 is illustrated from behind the head. The head of patient 251 may be in contacted with left side coil 556, right side coil 557, top coil 255, rear coil 558. Coils in this example are held in position by ball joints on frame 260, which is held in the proper overall position relative to patient 251 by frame suspension 265.

As mentioned above, the methods and systems described herein may involve the stimulation of the circuit of Papez, shown diagrammatically in FIG. 4. The circuit of Papez is one of the first brain circuits identified, and was described by James Papez in 1937. Signals are believed to travel from the cingulate to the hippocampus, then back to the cingulate. This circuit is one of the major pathways of the limbic system for cortical control of emotion, and is known to play a role in storing memory. In the context of the methods described herein, and without being bound by theory, the circuit of Papez may provide an electrical “highway” (path) between the cingulate and the hippocampus by which the therapeutic effects of the described method may be achieved.

FIG. 4 illustrates the known neuroanatomical Circuit of Papez, which provides the specific neuro-anatomical complex of structures upon which the present device and method are applied. These include backward-projecting neurons of cingulate 410, 411, which lead to projections into the entorhinal cortex 420, and projections into hippocampus 430. Also shown are the mammilothalamic tract 450, by which, hippocampal signals are relayed to the thalamus, and projections 460, 461, 462 from anterior nucleus of thalamus to back to the cingulate cortex.

Projections into the fornix 440 are also shown. Driving current flow retrograde through this connection is the manner in which neurosurgically (invasively) implanted with electrodes have been used in order to relay impulses to the entorhinal cortex and hippocampus.

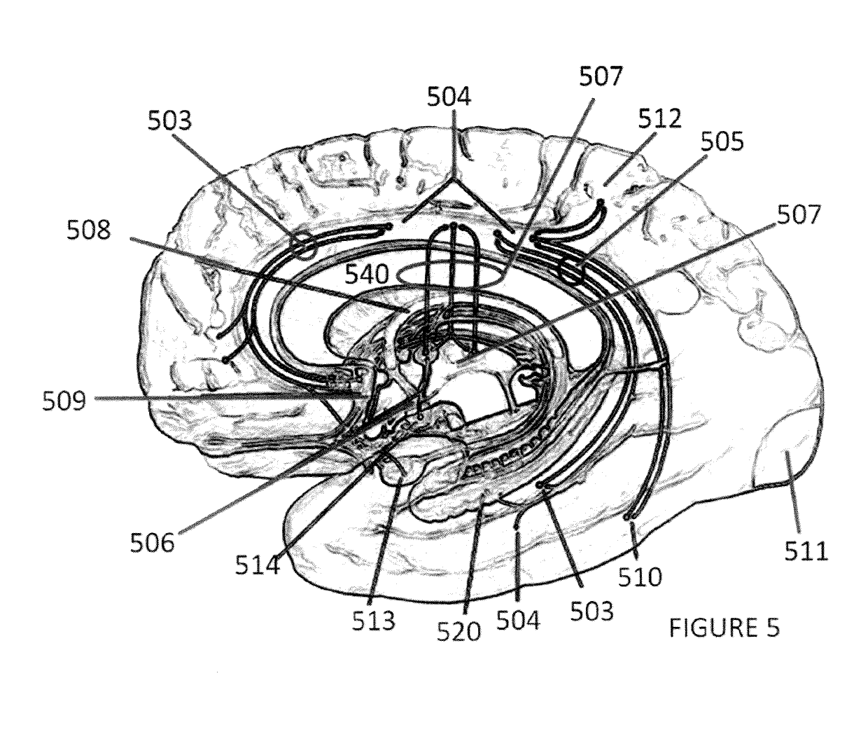

FIG. 5 illustrates the normal pathways that tie the cingulate to other brain regions, (including the circuit of Papez described in FIG. 4), plus additional connections with cingulate 504, the combination of the cingulate gyms (gray matter) plus cingulate bundle, or cingulum (white matter). The figure is similar to that shown in FIG. 1, but the areas affected by Alzheimer's disease early in the process are not graphically shown, thereby revealing a better picture of posterior cingulate bundle 505 and its afferent and efferent projections into the temporal lobe. Such projections include projections from posterior cingulate 505 into cingulate-temporal association areas tract 510, projections from posterior cingulate 505 to the entorhinal cortex 504, projections from posterior cingulate 505 to the subiculum 503 and projections from posterior cingulate 505 into entorhinal cortex 504 and hippocampus 520. These lateral areas, as shown in FIG. 1, are affected early in the course of development of Alzheimer's disease. Also shown are Amygdala 513, thalamo-cingulate connections 507, Corpus callosum 540, Septal nuclei 509, Fornix 508, Visual cortex 511, and mammillothalamic tract 506.

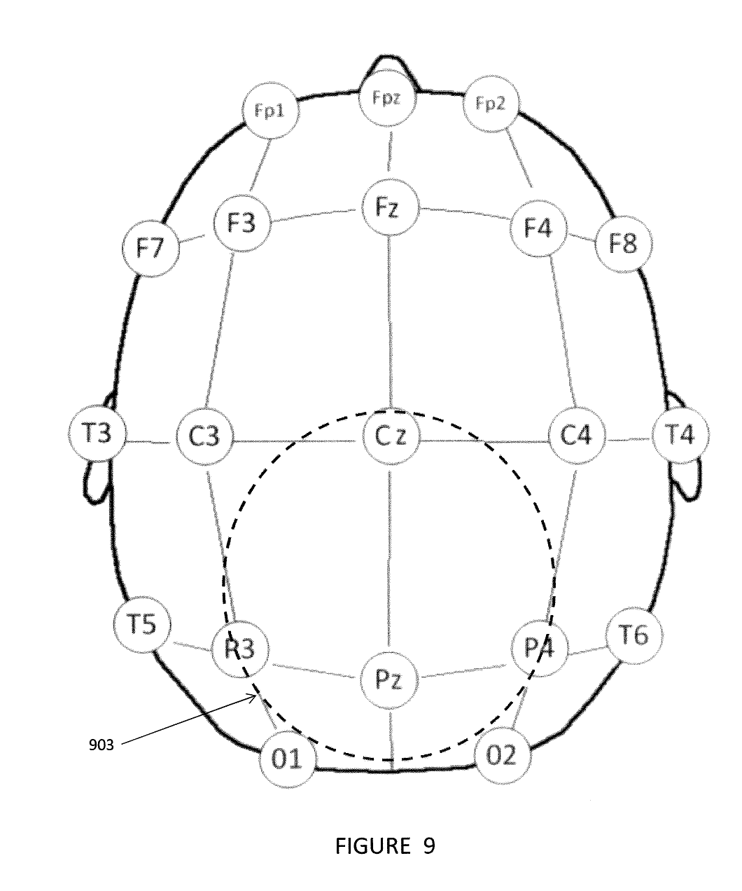

In general, the approach described herein includes, as illustrated in FIGS. 2 and 3, the use of multiple TMS coils that can be selectively placed a predetermined positions (e.g., see FIG. 9) around and/or against a subject's head and may be individually powered. This may allow the TMS coils to be individually steered and adjusted to induce electrical currents in a desired location, providing selectivity and flexibility in positioning the coils. The methods described herein may be used alone or in conjunction with other therapies (such as pharmaceutical therapies for Alzheimer's). Once positioned about the head, as described herein, a rapid (e.g. 10 Hz or greater) pulse pattern may be applied by the TMS coils to treat the patient. Treatment duration may be short (e.g., between 500 ms and 10 min) or longer (10 min-3 hrs, including 10 min, 15 min, 20 min, 25 min, 30 min, etc. Sessions may be repeated over time (e.g., every day, multiple times per day, multiple times per week, etc.) for as many sessions as desired (e.g. 30 session, 60 session, 90 sessions, etc.) Preliminary work suggests that multiple session may be beneficial to promoting a long-term effect, with persistence of the effect lasting greater than 3 months, (e.g., six months) or longer.

FIG. 6 is a 15-O PET image derived from use of a system including a coil array similar to that shown in FIGS. 2 and 3, placed over the anterior portion of the cingulate rather than the posterior portion of the cingulate. This image demonstrates significant selective perigenual anterior cingulate modulation 605 and relatively little modulation of the overlying cortical surface. It also displays an area of posterior cingulate modulation 610, probably of secondary, relayed origin. This PET image was produced using the coils arranged in a “configuration A” around a front region of the subject's head as described previously (and incorporated by reference) and suggests that that coil configuration A may be capable of relaying stimulation to the hippocampus and entorhinal cortex itself, even though the primary sit of modulation is anterior rather than posterior cingulate.

FIG. 7 shows an 15-O PET image derived from use of a system including a coil array identical to that shown in FIGS. 2 and 3, placed over the anterior portion of the cingulate (referred to as “Configuration B) rather than the posterior portion of the cingulate as shown in FIGS. 2 and 3. This image demonstrates significant selective perigenual anterior cingulate modulation 705 (arrow) an relatively little cortical surface modulation. It also displays small area of posterior cingulate modulation 710. FIG. 8 is a schematic illustrating the symmetry in the anterior portion 805 and posterior portion 810 of the cingulate bundles of the brain, and similar features of overlying superficial cortex.

As shown in FIGS. 2 and 3 and in FIG. 9, below, the arrangement of the TMS coils around the posterior (back) of the subject's head, and particularly around the C_{Z}, P₃, P₂, and P₄ standard EEG 10-20 system set of locations may result in an especially potent effect. FIG. 9 shows a standard EEG 10-20 system set of locations on a generic patient's head, allowing reproducible placement of the TMS coils using this reference system. In particular, FIG. 9 illustrates a region 903 including the C_{Z}, P₃, P₂, and P₄ regions that may be used to position a plurality of the TMS coils, as shown in FIGS. 2 and 3 for treatment.

In some variations, the methods described herein may include the use of only a single TMS coil positioned in the posterior region of the subject's head (e.g., the portion including the C_{Z}, P₃, P₂, and P₄ regions illustrated in FIG. 9). In this embodiment a second TMS coil may be positioned elsewhere (e.g., outside of this region) or may not be present at all; other aspects of the TMS applied, such as the use of rapid TMS pulsing at greater than 10 Hz, the duration of stimulation and the use of repeated treatments, may be the same as described above. For example, a method for treating Alzheimer's may include: orienting a transcranial magnetic stimulation (TMS) coil over or against a patient's head within a region including the C_{Z}, P₃, P₂, and P₄ standard EEG 10-20 system set of locations to direct TMS stimulation towards the patient's posterior cingulate; applying a rapid TMS pulse pattern of at least 10 Hz from the TMS coil to drive stimulation of the patient's posterior cingulate for about thirty seconds or more; an repeating the orienting and applying steps above for a plurality of treatment sessions.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

We claim:

1. A method for treating Alzheimer's by non-invasively stimulating brain areas involved in Alzheimer's disease pathology, the method comprising:
orienting a plurality of transcranial magnetic stimulation (TMS) coils around a back of a patient's head within a region including the C_{Z}, P₃, P₂, and P₄ standard EEG 10-20 system set of locations to direct TMS stimulation towards the patient's posterior cingulate;
applying a rapid TMS pulse pattern of at least 10 Hz from each of the plurality of TMS coils to drive stimulation of the patient's posterior cingulate for about thirty seconds or more; and
repeating the orienting and applying steps above for a plurality of treatment sessions.

2. The method of claim 1, wherein repeating comprises repeating more than 30 sessions.

3. The method of claim 1, wherein repeating comprises repeating more than 60 sessions.

4. The method of claim 1, wherein orienting comprises positioning between three and six TMS coils around the patient's head at different locations to apply TMS that stimulates the subject's posterior cingulate.

5. The method of claim 1, wherein orienting comprises positioning a first TMS coil and a second TMS coil against or adjacent to a region of the patient's head including the C_{Z}, P₃, P₂, and P₄ standard EEG 10-20 system set of locations.

6. The method of claim 1, wherein applying a rapid TMS pulse pattern comprises stimulating the posterior cingulate and efferents going to the patient's subiculum and dentate regions.

7. The method of claim 1, wherein applying comprises independently applying pulsed TMS from each of two or more TMS coils oriented around a back region of the subject's head to evoke stimulation of the subject's posterior cingulate.

8. The method of claim 1, wherein applying comprises applying pulsed TMS at at least 10 Hz from each of three or more TMS coils oriented around a back region of the subject's head to neuromdulate the subject's posterior cingulate nerve fiber bundle until action potentials are propagated into the subject's entorhinal cortex and hippocampus to increase neuronal activity therein.

9. The method of claim 1, wherein positioning comprises locking two or more TMS coils into a position around a back of the subject's head to aim stimulation from the plurality of TMS coils to the subject's posterior cingulate nerve fiber bundle.

10. The method of claim 1, wherein applying the rapid TMS pulse pattern produces a net increase in metabolic activity in the subject's posterior cingulate fiber bundle.

11. The method of claim 1, wherein applying the rapid TMS pulse pattern produces an increase in metabolic activity in the temporal lobe.

12. The method of claim 1, wherein applying the rapid TMS pulse pattern from each of the plurality of TMS coils comprises applying a rapid TMS pulse pattern for between about one minute and 1 hour.

13. The method of claim 1, wherein positioning further comprises positioning the TMS coils with respect to the patient wherein the patient displays a mild cognitive impairment to early dementia.

14. A method for treating Alzheimer's by non-invasively stimulating brain areas involved in Alzheimer's disease pathology, the method comprising:
positioning a first transcranial magnetic stimulation (TMS) coil and a second TMS coil against or adjacent to a back of a patient's head within a region including the C_{Z}, P₃, P₂, and P₄ standard EEG 10-20 system set of locations on the patient's head;
applying a rapid TMS pulse pattern from each of the plurality of TMS coils to drive stimulation of the patient's posterior cingulate for between about thirty seconds and 3 hours; and
repeating the orienting and applying steps above for a plurality of treatment sessions.

15. The method of claim 14, wherein positioning further comprises positioning a third TMS coil against or adjacent to the back of a patient's head.

16. The method of claim 14, wherein applying the rapid TMS pulse pattern comprises applying a rapid TMS pulse pattern having a frequency of greater than 10 Hz.

17. The method of claim 14, wherein applying a rapid TMS pulse pattern comprises applying pulses of TMS from each of the plurality of TMS coils.

18. The method of claim 14, wherein applying a rapid TMS pulse pattern comprises producing a net increase in metabolic activity in the posterior cingulate.

19. The method of claim 14, wherein applying a rapid TMS pulse pattern comprises increasing metabolic activity in the patient's temporal lobe.

20. A method for treating Alzheimer's by non-invasively stimulating brain areas involved in Alzheimer's disease pathology, the method comprising:
positioning a first transcranial magnetic stimulation (TMS) coil, a second TMS coil, and a third TMS coil against or adjacent to a back of a patient's head within a region including the C_{Z}, P₃, P₂, and P₄ standard EEG 10-20 system set of locations on the patient's head;
applying a rapid TMS pulse pattern from each of the plurality of TMS coils for greater than about thirty seconds;
increasing metabolic activity in the posterior cingulate; and
repeating the orienting and applying steps above for a plurality of treatment sessions.
